(19) United States
(12) Patent Application Publication
(10) Pub. No.: US 2004/0256480 A1
(43) Pub. Date: Dec. 23, 2004

(54) An air freshening or purifying sheet

(76) Inventors: Robert Vern Channer, Oxon (GB); Paul Robinson, Hull (GB)

Correspondence Address: NORRIS, MCLAUGHLIN & MARCUS, 875 THIRD AVE, 18TH FLOOR, NEW YORK, NY, 10022, US

(21) Appl. No.: 10/479,017

(22) PCT Filed: May 24, 2002
(86) PCT No.: PCT/GB02/02445

(30) Foreign Application Priority Data
May 29, 2001 (GB) 0112925.3

(51) Int. Cl. A61L 9/04

(52) U.S. Cl. 239/34

(57) ABSTRACT

The invention relates to improvements in or relating to air freshening or purifying devices in the form of sheets. The invention therefore provides an air freshening or purifying sheet comprising a substrate in the form of a sheet having opposing surfaces, wherein a releasable adhesive is applied to at least one region of one of said surfaces, and at least another region of one or both of said surfaces being coated with a layer of an air freshening or purifying composition in which the said composition is a gel composition resulting from the cross-linking in situ, of a homopolymer or copolymer in the presence of a perfuming, deodorising or insecticidal base.

The invention relates to improvements in or relating to air freshening or purifying devices in the form of sheets.

Air freshening and purifying devices are used in many forms, such as plastic containers, jars, impregnated rings and the like. These contain, or are impregnated with, a fragrance which evaporates over a period of time and is released into the atmosphere. In this specification the term “fragrance” may be a simple perfume, but also includes a deodorant which itself may or may not be perfumed. The term “air purifying” is intended to include compositions having disinfecting and insecticidal capabilities.

Such device are typically bulky as they are intended to be used for a reasonable period of time, typically several weeks or months, and therefore need to include sufficient fragrance to last for that period of time. Such devices also usually require a surface on which to sit undisturbed or a place from which to be hung.

However there is a need for shorter term air fresheners, and ones that can be placed anywhere.

It is therefore an object of the present invention to overcome these disadvantages.

According to the invention there is provided an air freshening or purifying sheet comprising a substrate in the form of a sheet having opposing surfaces, wherein a releasable adhesive is applied to at least one region of a first of said surfaces, and at least another region of one or both of said surfaces is coated with a layer of an air freshening or purifying composition in which the said composition is a gel composition resulting from the cross-linking in situ, of a homopolymer or copolymer in the presence of a perfuming, deodorising or insecticidal base, said layer being of a thickness of less than 1 mm such that the coated surface is able to receive printing or writing.

There is also provided a collocation of air freshening or purifying sheets as defined above in which a plurality of said sheets are releasably held together so that one sheet at a time may be removed from the collocation.

A preferred embodiment of the present invention will now be described by way of example only, with reference to the accompanying drawings in which:—

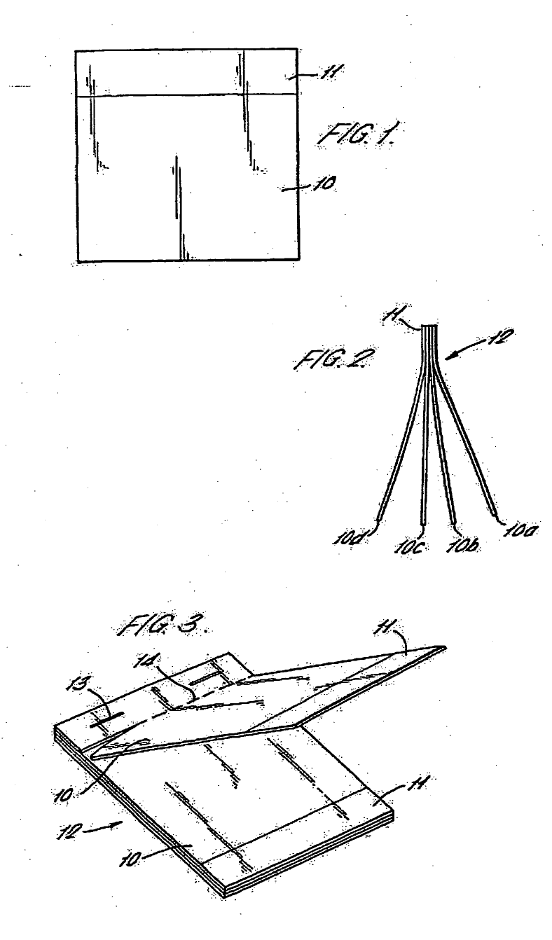

FIG. 1 is a rear elevation of an air freshening sheet according to the present invention;

FIG. 2 is a side elevation of a collocation of air freshening sheets in the form of a booklet; and

FIG. 3 is a plan view of an alternative form of booklet.

Referring first to FIG. 1 there is shown an air freshening sheet 10 comprising a substrate formed from a thin sheet of material having a thickness of from 0.1 to 4 mm, especially 0.5 to 2 mm. They material may be a permeable or porous material such as paper or card or an impermeable plastic material. Although the drawings illustrate a square sheet 10, the actual shape of the sheets 10 is not important. The sheet 10 may conveniently be of the size 2 cm×7 cm, although any convenient size may be used.

On one surface of the sheet 10 there is provided a layer of a releasable adhesive 11, either on the whole of the surface or in a limited region. This may be along one edge as illustrated, or the adhesive region 11 may be provided at any location on the sheet 10. Alternatively a plurality of small adhesive regions may be used on each sheet 10. The adhesive 11 may be a standard contact adhesive, which may be releasably attached to a desired surface such as a wall or dashboard of a car.

At least a second region of the sheet 10, if not the whole sheet 10, is coated with a thin layer of an air freshening (fragrancing or air purifying) gel. The second region(s) may be on one or both sides of the sheet 10. The gel may be as described in, for example, U.S. Pat. No. 5,780,527. Thus it is, for example, a gel resulting from the cross-linking, in situ of a homopolymer or copolymer in the presence of a perfuming, deodorising or insecticidal base. A suitable copolymer is maleinised polybutadiene or polyisoprene such as Lithene, N4-9000 10MA (Registered Trade Mark) obtainable from Revertex Limited. Suitable cross-linking agent, for example a cocoamine having 5 oxyethylene units per molecule sold under the name Crodamet (Registered Trade Mark) obtainable from Croda Chemicals Limited.

The gel composition may be applied to the surface of the sheets 10 by a dipping or spreading process. The quantity of the composition applied is selected to provide a relatively short term dissipation of, say, three days to four weeks, for example about one week. It is envisaged that a coating of less than 1 mm thickness would be suitable, and probably of less than 0.1 mm. If the sheet 10 is to receive printed information or indicia, or is to be handwritten upon, the layer of gel needs to be on the thinner side. If the layer is too thick, the sheet 10 does not receive printing or writing.

Where the substrate is impermeable, the gel remains in a layer on the surface thereof. Where the substrate is permeable some of the fragrancing, deodorising or insecticidal base leaks out of the gel layer into the substrate. This can be advantageous if the gel layer is applied to one surface of the substrate only, as the fragrancing (or other) effect then emanates from both surfaces.

The sheet 10 can be applied to any convenient surface such as a wall, shelf, window sill, an item of furniture or car dashboard etc., simply by placing the region bearing the adhesive 11 into contact with the surface. When the air freshening composition has dissipated or the air freshening action is no longer required the sheet can simply be peeled from the surface to which it has been applied, and disposed of.

Such air freshening sheets 10 can conveniently be supplied in a number of different formats. Once such format is shown in FIG. 2, whereby a collocation of sheets 10a, b, c, d are placed together in a coherent manner to form a booklet 12, such that the adhesive 11 on one sheet 10a adheres to an adjacent sheet 10b. When a sheet 10 is required for use, the top one is simply peeled from the booklet 12, leaving the remaining sheets 10 for future use.

An alternative form is illustrated in FIG. 3 in which a number of sheets are securely held together at one end by staples 13, or other suitable means. When a sheet 10 is required for use the top one is removed by tearing it along a perforated line 14. If the adhesive regions 11 are provided on the sheets 10 at the opposite end to the staples 13, this helps to keep the leaves of the booklet 12 closed.

In a further alternative arrangement, a plurality of sheets 10 can be provided on discrete sheets of a carrier layer, or even a continuous elongate carrier layer, e.g. waxed paper, in a similar manner to the way in which labels are provided.

In yet another embodiment a single elongate strip may be provided e.g. in a roll, with a plurality of sheets 10 separated by a row of perforations. Each sheet 10 can be torn off as and when requited, in a similar manner to toilet tissues.

In yet another embodiment of the invention, each air freshening sheet 10 within a booklet 12 is separated by a non-air freshening layer. This has the advantage that as the top sheet 10 is removed from the booklet 12, the next sheet is not immediately exposed to the atmosphere. The non-air freshening layers are conveniently provided by waxed paper or an impermeable substrate.

The air freshening sheets 10 may also be provided in a form where each sheet is individually wrapped in an impermeable envelope. In this embodiment, the envelopes may be securely bound or bonded together to form a booklet and each envelope can be torn off from the booklet.

The air freshening composition may comprise a fragrance, a disinfectant or an insecticide.

1. An air freshening or purifying sheet comprising a substrate in the form of a sheet having opposing surfaces, wherein a releasable adhesive is applied to at least one region of a first said surfaces, and at least another region of one or both of said surfaces is coated with a layer of an air freshening or purifying composition in which the said composition is a gel composition resulting from the cross-linking in situ, of a homopolymer or copolymer in the presence of a perfuming, deodorising or insecticidal base, said layer being of a thickness of less than 1 mm such that the coated surface is able to receive printing or writing.

2. An air freshening or purifying sheet according to claim 1 in which said gel composition is applied to at least a region of a second of said surfaces of the substrate.

3. An air freshening or purifying sheet according to claim 1 in which the releasable adhesive is applied to a plurality of regions of the surface of the substrate.

4. An air freshening or purifying sheet according to claim 1 in which the releasable adhesive is applied to the whole of one surface of the substrate.

5. A collocation of air freshening or purifying sheets according to claim 1 in which a plurality of said sheets are releasably held together so that one sheet at a time may be removed from the collocation.

6. A collocation of air freshening or purifying sheets according to claim 5 in which the sheets are held together by the adhesive regions.

7. A collocation of air freshening or purifying sheets according to claim 5 in which the sheets are held together by mechanical means.

8. A collocation of air freshening or purifying sheets according to claim 7 in which the mechanical means comprise staples.

9. A collocation of air freshening or purifying sheets according to claim 5 in which each sheet is provided with perforations, enabling the sheet to be torn from the collocation.

10. A collocation of air freshening or purifying sheets according to claim 5 in which the sheets are separated by a non-air freshening sheet.

11. A collocation of air freshening or purifying sheets according to claim 5 in which each sheet is individually wrapped in an impermeable envelope and the envelopes are secured together such that each envelope can be removed from the collocation one at a time and the sheet removed from the envelope.

12. A collocation of air freshening or purifying sheets according to claim 1 provided in a container.

13. A collocation of air freshening or purifying sheets according to claim 12 claim 12 in which each sheet is individually wrapped in an impermeable envelope.

14. An air freshening or purifying sheet according to claim 2 in which the releasable adhesive is applied to a plurality of regions of the surface of the substrate.

15. An air freshening or purifying sheet according to claim 2 in which the releasable adhesive is applied to the whole of one surface of the substrate.

16. A collocation of air freshening or purifying sheets according to claim 2 in which a plurality of said sheets are releasably held together so that one sheet at a time may be removed from the collocation.

17. A collocation of air freshening or purifying sheets according to claim 16 in which the sheets are held together by the adhesive regions.

18. A collocation of air freshening or purifying sheets according to claim 16 in which the sheets are held together by mechanical means.

19. A collocation of air freshening or purifying sheets according to claim 16 in which the sheets are separated by a non-air freshening sheet.

20. A collocation of air freshening or purifying sheets according to claim 16 in which each sheet is individually wrapped in an impermeable envelope and the envelopes are secured together such that each envelope can be removed from the collocation one at a time and the sheet removed from the envelope.
